(19) United States
(12) Patent Application Publication
(10) Pub. No.: US 2021/0330023 A1
(43) Pub. Date: Oct. 28, 2021

(54) SHOES FOR MASSAGING THE INNER SIDE OF HALLUX

(71) Applicant: Guangdong Breath Walker Health Technology Co., Ltd., Dongguan City (CN)

(72) Inventors: Xingliang WU, Dongguan City (CN); Yuyuan ZHOU, Dongguan City (CN); Kanglong LIU, Dongguan City (CN)

(21) Appl. No.: 17/186,557

(22) Filed: Feb. 26, 2021

(30) Foreign Application Priority Data
Apr. 28, 2020 (CN) 202020677581.3

(51) Int. Cl. A43B 7/14

(52) U.S. Cl. CPC A43B 7/146

(57) ABSTRACT

A shoe for massaging the inner side of hallux includes a sole, a vamp mounted on the sole, and an elastic correction band with both ends installed to the inner side of the vamp and the top of the sole respectively. The elastic correction band has a massage area on an outer surface of the hallux and massage bumps on the massage area. Both ends of the elastic correction band have a fixed part on the inner side of the vamp, and the fixed part at the lower end of the elastic correction band is installed to the top of the sole. The massage area of the elastic correction band can massage the trigeminal nerve acupoints on the inner side of the hallux to achieve the effect of relieving migraine. In addition, the elastic correction band can correct the hallux valgus and provide a certain buffering effect by its elasticity.

### FIELD OF THE INVENTION

The present invention relates to shoes, and more particularly to the shoes for massaging the inner side of hallux.

### BACKGROUND OF THE INVENTION

At present, massage can be divided into head massage, neck massage, waist massage, back massage, buttock massage, foot massage, etc. Among these massages, foot massage plays an important role in people's health and regular medical healthcare, but most foot massages are focused on the soles of the feet, while the massage between the toes is ignored.

In addition, hallux valgus is one of the most common diseases of the foot, and it is also the most common disease of the hallux (or big toe). Hallux valgus is primarily represented by the position of the big toe deviating outward to have an angle greater than 15° between the proximal phalanx of hallux and the first metatarsal. The most common problems caused by hallux valgus include pain and difficulty in wearing shoes. In severe cases, hallux valgus can also cause arthritis and degenerative changes in the hallux metatarsophalangeal joint; and it can also induce deformity and dislocation of the second toe.

However, the existing surgical treatments for correcting hallux valgus has the drawbacks of complicated operation, trauma of patients' body, and long recovery time, which will cause lots of inconvenience to people's body, mind, and life. Existing functional shoes cannot prevent and correct the condition of hallux valgus and will aggravate the situation of hallux valgus and affect the relief of hallux valgus.

### SUMMARY OF THE INVENTION

It is a primary objective of the present invention to provide a pair of shoes for massaging the inner side of a hallux in order to solve the problems that the traditional massage shoes cannot massage the inner side of the hallux or prevent and correct hallux valgus, and the existing surgical correction procedures for the treatment of hallux valgus is complicated.

To achieve the aforementioned and other objectives, the present invention discloses a shoe for massaging the inner side of hallux, comprising a sole 10, a vamp 20 mounted on the sole 10, and an elastic correction band 30, and both ends of the elastic correction band 30 are installed to an inner side of the vamp 20 and a top side of the sole 10; the elastic correction band 30 has a massage area 31 defined in the middle of an outer surface on a side of the hallux, and a plurality of massage bumps 311 formed on the massage area 31; the elastic correction band 30 further has a fixed part 32 disposed on both sides of the massage area 31, and the fixed part 32 at the upper end of the elastic correction band 30 is fixedly installed to an inner side of the vamp 20, and the fixed part 32 at the lower end of the elastic correction band 30 fixedly installed to a top side of the sole 10.

Further, the sole 10 comprises an outsole 11 and a Laban insole 12 installed on a top side of the outsole 11, and the Laban insole 12 has a via 121 formed thereon, and a bottom end of the elastic correction band 30 is passed through the via 121 and then folded and fixed to a back side of the Laban insole 12.

In summation, the elastic correction band 30 of the present invention not just can massage the inner side of hallux to achieve the effect of relieving migraine only, but also can provide the effect of correcting hallux valgus while providing a certain buffering effect by the elasticity of the elastic correction band 30 to avoid foot pain caused by forced correction and prevent increased valgus. The elastic correction band 30 of this invention has the features of simple structure, convenient installation, high practicality and strong promotional effect.

### BRIEF DESCRIPTION OF THE DRAWINGS

FIG. 1 is a schematic view of a massage shoe for the inner side of hallux in accordance with the present invention;

FIG. 2 is a cross-sectional view of Section A-A of FIG. 1; and

FIG. 3 is a schematic view showing an unfolded structure of an elastic correction band as depicted in FIG. 2.

### DESCRIPTION OF THE PREFERRED EMBODIMENTS

The objectives, technical characteristics and advantageous effects of the present invention will become apparent with the detailed description of preferred embodiments accompanied with the illustration of related drawings. It is intended that the embodiments and drawings disclosed herein are to be considered illustrative rather than restrictive.

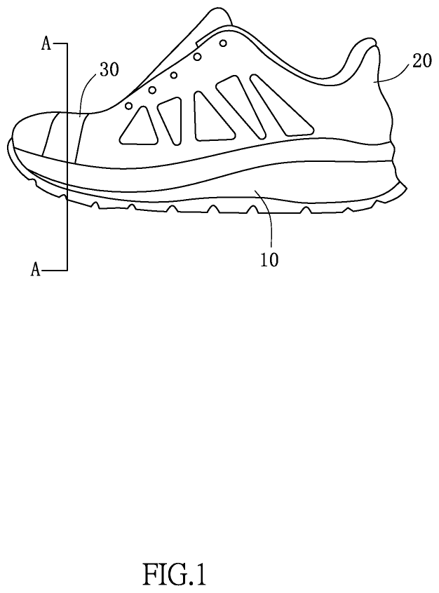

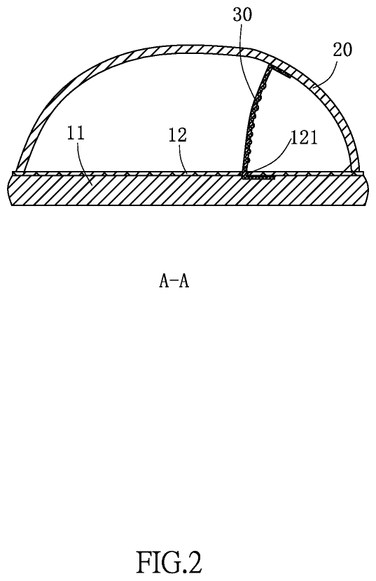

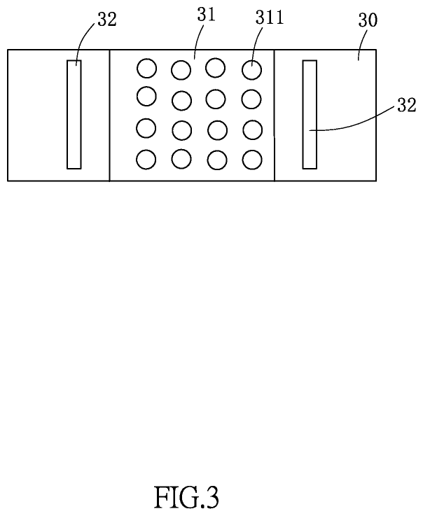

With reference to FIGS. 1 to 3 for a massage shoe for the inner side of hallux in accordance with the present invention, the shoe comprises a sole 10, a vamp 20 mounted on the sole 10, and an elastic correction band 30 with both ends installed to an inner side of the vamp 20 and the top side of the sole 10.

The sole 10 comprises an outsole 11 and a Laban insole 12 disposed on a top side of the outsole 11, and the Laban insole 12 has a via 121 formed thereon. The elastic correction band 30 has a massage area 31 defined at the middle of the elastic correction band 30, and a plurality of massage bumps 311 formed on the massage area 31. The elastic correction band 30 has a fixed part 32 disposed separately on both left and right sides of the massage area 31, and the two fixed parts 32 can be fixed onto a bottom side of the Laban insole 12 and an inner side of the vamp 20 by pasting, sewing or high-temperature heat pressing.

During installation, the elastic correction band 30 is installed between the hallux and the second toe, and the side of the elastic correction band 30 with the massage bumps 311 is configured to be facing the hallux. The fixed part 32 at the top end of the elastic correction band 30 is fixedly installed onto the inner side of the vamp 20, and a bottom end of the elastic correction band 30 is passed through the via 121 of the Laban insole 12 and then folded and fixed onto a bottom side of the Laban insole 12.

In use, the massage area 31 of the elastic correction band 30 can massage the trigeminal nerve acupoints on the inner side of the hallux to achieve the effect of relieving migraine. In addition, the elastic correction band 30 can separate the hallux from the second toe to correct the hallux valgus and provide a certain buffering effect by its elasticity, so as to avoid foot pain caused by forced correction and prevent increased valgus. This invention has high practicality and strong promotional effect.

In summation of the description above, the elastic correction band 30 of the present invention not just can massage the inner side of hallux to achieve the effect of relieving migraine only, but also can provide the effect of correcting hallux valgus while providing a certain buffering effect by the elasticity of the elastic correction band 30 to avoid foot pain caused by forced correction and prevent increased valgus. The elastic correction band 30 of this invention has the features of simple structure, convenient installation, high practicality and strong promotional effect.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A shoe for massaging the inner side of hallux, comprising: a sole, a vamp mounted on the sole, and an elastic correction band with both ends installed to an inner side of the vamp and a top side of the sole respectively; the elastic correction band comprising a massage area defined in the middle of an outer surface of a side of the hallux, and a plurality of massage bumps disposed on the massage area; the elastic correction band comprising a fixed part disposed separately on both sides of the massage area, and the fixed part at an upper end of the elastic correction band being fixedly installed onto an inner side of the vamp, and the fixed part at a lower end of the elastic correction band being fixedly installed onto a top side of the sole.

2. The shoe for massaging the inner side of hallux as claimed in claim 1, wherein the sole comprises an outsole, a Laban insole installed onto a top side of the outsole, a via formed on the Laban insole, and a bottom end of the elastic correction band passing through the via and being folded and fixed to a back side of the Laban insole.
